(19) United States
(12) Patent Application Publication
(10) Pub. No.: US 2010/0312169 A1
(43) Pub. Date: Dec. 9, 2010

(54) Method of operating a vitrectomy probe

(76) Inventors: Jack R. Auld, Laguna Niguel, CA (US); John C. Huculak, Mission Viejo, CA (US)

Correspondence Address: ALCON, IP LEGAL, TB4-8, 6201 SOUTH FREEWAY, FORT WORTH, TX, 76134, US

(21) Appl. No.: 12/567,919

(22) Filed: Sep. 28, 2009

Related U.S. Application Data
(60) Provisional application No. 61/183,762, filed on Jun. 3, 2009.

(51) Int. Cl. A61F 9/007

(52) U.S. Cl. 604/22

(57) ABSTRACT

An improved method of operating a vitrectomy probe is disclosed in which the amount of time that the probe port is open during each cut cycle of the probe is fixed and minimized over all cut rates of the probe.

This application claims the priority of U.S. Provisional Application No. 61/183,762 filed Jun. 3, 2009.

### FIELD OF THE INVENTION

The present invention pertains to vitrectomy probes. More particularly, but not by way of limitation, the present invention pertains to an improved method of operating vitrectomy probes in ophthalmic surgery.

### DESCRIPTION OF THE RELATED ART

Many microsurgical procedures require precision cutting and/or removal of various body tissues. For example, certain ophthalmic surgical procedures require the cutting and/or removal of the vitreous humor, a transparent jelly-like material that fills the posterior segment of the eye. The vitreous humor, or vitreous, is composed of numerous microscopic fibrils that are often attached to the retina. Therefore, cutting and removal of the vitreous must be done with great care to avoid traction on the retina, the separation of the retina from the choroid, a retinal tear, or, in the worst case, cutting and removal of the retina itself. In particular, delicate operations such as mobile tissue management (e.g. cutting and removal of vitreous near a detached portion of the retina or a retinal tear), vitreous base dissection, and cutting and removal of membranes are particularly difficult.

The use of microsurgical cutting probes in posterior segment ophthalmic surgery is well known. Such vitrectomy probes are typically inserted via an incision in the sclera near the pars plana. The surgeon may also insert other microsurgical instruments such as a fiber optic illuminator, an infusion cannula, or an aspiration probe during the posterior segment surgery. The surgeon performs the procedure while viewing the eye under a microscope.

Conventional vitrectomy probes typically include a hollow outer cutting member, a hollow inner cutting member arranged coaxially with and movably disposed within the hollow outer cutting member, and a port extending radially through the outer cutting member near the distal end thereof Vitreous humor and/or membranes are aspirated into the open port, and the inner member is actuated, closing the port. Upon the closing of the port, cutting surfaces on both the inner and outer cutting members cooperate to cut the vitreous and/or membranes, and the cut tissue is then aspirated away through the inner cutting member. U.S. Pat. Nos. 4,577,629 (Martinez); 5,019,035 (Missirlian et al.); 4,909,249 (Akkas et al.); 5,176,628 (Charles et al.); 5,047,008 (de Juan et al.); 4,696,298 (Higgins et al.); and 5,733,297 (Wang) all disclose various types of vitrectomy probes, and each of these patents is incorporated herein in its entirety by reference.

Conventional vitrectomy probes include “guillotine style” probes and rotational probes. A guillotine style probe has an inner cutting member that reciprocates along its longitudinal axis. A rotational probe has an inner cutting member that reciprocates around its longitudinal axis. In both types of probes, the inner cutting members are actuated using various methods. For example, the inner cutting member can be moved from the open port position to the closed port position by pneumatic pressure against a piston or diaphragm assembly that overcomes a mechanical spring. Upon removal of the pneumatic pressure, the spring returns the inner cutting member from the closed port position to the open port position. As another example, the inner cutting member can be moved from the open port position to the closed port position using a first source of pneumatic pressure, and then can be moved from the closed port position to the open port position using a second source of pneumatic pressure. As a further example, the inner cutting member can be electromechanically actuated between the open and closed port positions using a conventional rotating electric motor or a solenoid. U.S. Pat. No. 4,577,629 provides an example of a guillotine style, pneumatic piston/mechanical spring actuated probe. U.S. Pat. Nos. 4,909,249 and 5,019,035 disclose guillotine style, pneumatic diaphragm/mechanical spring actuated probes. U.S. Pat. No. 5,176,628 shows a rotational dual pneumatic drive probe.

Despite the above described advances, a need still exists for improved operation of vitrectomy probes. In particular, a method of operating vitrectomy probes that minimizes traction on the retina during vitrectomy, maximizes the rate at which vitreous and other tissue can be removed, enhances membrane dissection, and maximizes patient safety is particularly desired.

### SUMMARY OF THE INVENTION

In one aspect, the present invention is a method of operating a vitrectomy probe. A vitrectomy probe is provided that has a tubular outer cutting member with a port for receiving ophthalmic tissue, and a tubular inner cutting member disposed within the outer cutting member. The probe also has an actuating mechanism for reciprocating actuation of the inner cutting member so that the inner cutting member opens and closes the port and cuts ophthalmic tissue disposed in the port. The port of the vitrectomy probe is disposed in a posterior segment of an eye. Ophthalmic tissue is aspirated into the port. A computer and the actuating mechanism are used to operate the probe over a range of cut rates so that an amount of time that the port is open during each cut cycle of the probe is fixed and minimized over all cut rates within the range of cut rates.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a perspective view of a vitrectomy probe according to a preferred embodiment of the present invention;

FIG. 2 is a top view of the probe of FIG. 1;

FIG. 3 is a side, sectional view of the probe of FIG. 1 shown operatively coupled to a schematic of a microsurgical system;

FIG. 4 is an enlarged, perspective view of the cam member of the probe of FIG. 1;

FIG. 5 is a cross-sectional view of the cam member of FIG. 4;

FIG. 6 is an enlarged, fragmentary, side, sectional view of the portion of the probe of FIG. 1 shown in circle 6 of FIG. 2;

FIG. 7 is an enlarged, fragmentary, side, sectional view of a portion of the actuating mechanism of the probe of FIG. 1;

FIGS. 8A and 8B are schematic illustrations of the different characteristic lengths of cut ophthalmic tissue for conventional operation of a vitrectomy probe (FIG. 8A) and the method of operating a vitrectomy probe according to the present invention (FIG. 8B); and

FIG. 9 is a preferred illustration of duty cycle vs. cut rate for the method of operating a vitrectomy probe according to the present invention.

### DETAILED DESCRIPTION OF PREFERRED EMBODIMENTS

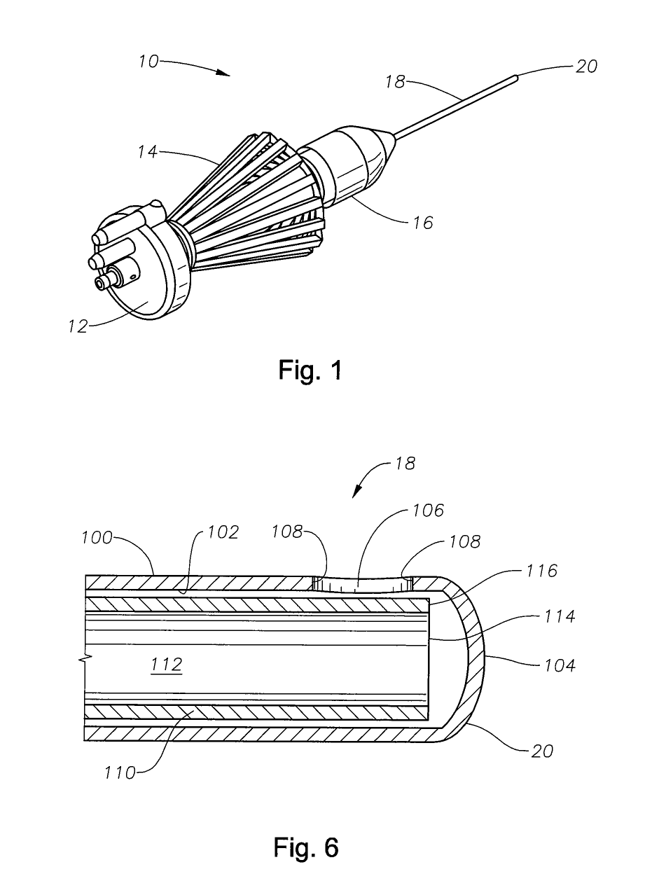

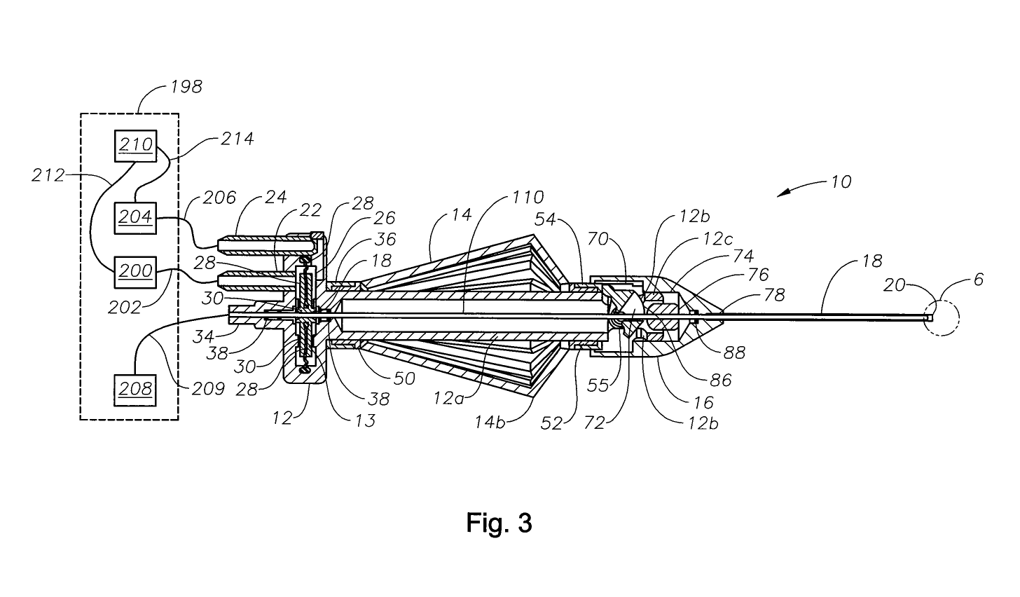

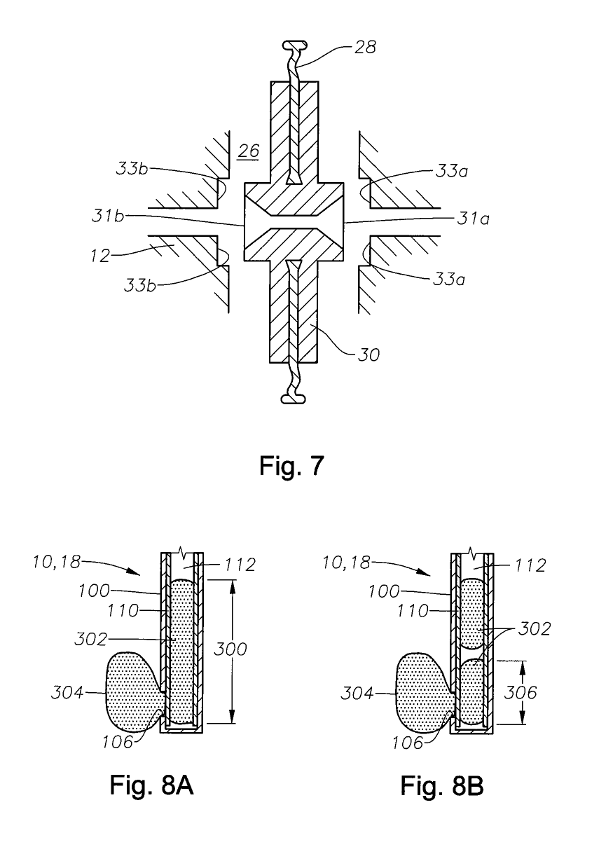

The preferred embodiments of the present invention and their advantages are best understood by referring to FIGS. 1 through 9 of the drawings, like numerals being used for like and corresponding parts of the various drawings.

Vitrectomy probe 10 preferably includes a base 12, an actuating handle 14, a nose member 16, and a cutting member 18 having a distal tip 20. Base 12 includes an actuating mechanism 13 for actuating a tubular inner cutting member 110 of cutting member 18 in a reciprocating manner. Actuating mechanism 13 preferably includes a first pneumatic port 22, a second pneumatic port 24, a diaphragm chamber 26, a flexible diaphragm 28, and a rigid center support 30. Flexible diaphragm 28 is coupled to center support 30 and base 12. As shown in the Figures, flexible diaphragm is frictionally coupled to both center support 30 and base 12. Alternatively, flexible diaphragm 28 may be frictionally coupled to base 12 and over-molded onto center support 30. Center support 30 has limiting surfaces 31a and 31b for interfacing with wall portions 33a and 33b of diaphragm chamber 26, respectively. Base 12 further includes an aspiration port 34 and a distal portion 12a having an aperture 12b and a distal tip 12c. A collar 36 couples distal portion 12a to actuating handle 14. Inner cutting member 110 is coupled to center support 30 and is slidaby and fluidly coupled to base 12 via o-rings 38.

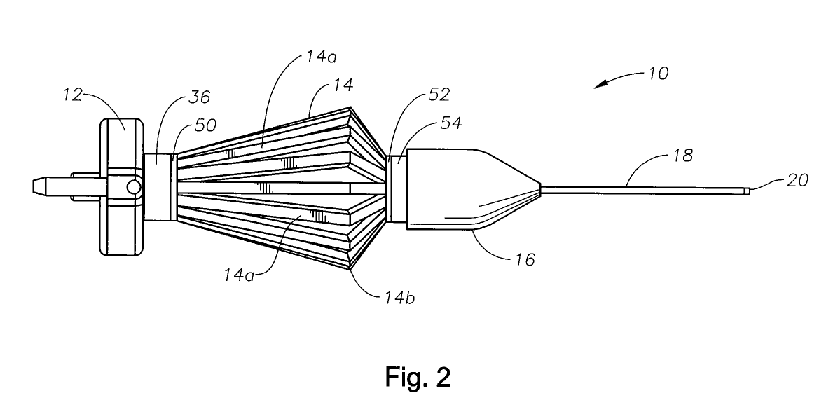

As shown in the Figures, actuating handle 14 includes a proximal base 50, a distal base 52, and a plurality of flexible appendages 14a coupled to both base 50 and 52. Flexible appendages 14a may be made from any suitable springy material having a memory, such as titanium, stainless steel, or a suitable thermoplastic. Handle 14 surrounds distal portion 12a of base 12. Proximal base 50 is coupled to collar 36. Distal base 52 is received within a slidable collar 54. A user grasps microsurgical instrument 10 via handle 14. When a user exerts an inward pressure on flexible appendages 14a, flexible appendages 14a bend at or near 14b, straightening and elongating flexible appendages 14a, and moving collar 54 toward distal tip 20. When such pressure is removed, spring 55 returns flexible appendages 14a to the position shown in FIG. 2.

As shown in the Figures, nose member 16 preferably includes cam chamber 70 for receiving a cam member 72, a base chamber 74 for receiving distal tip 12c of base 12, a bushing 76 for receiving inner cutting member 110 of cutting member 18, and an outlet 78 for receiving a tubular outer cutting member 100 of cutting member 18. Cam member 72 is rotationally coupled to nose member 16 within aperture 12b of base 12 via dowel pins (not shown) inserted into each end of a bore 79. Cam member 72 preferably has a first stopping surface 80 for interfacing with collar 54, a second stopping surface 82 for interfacing with base 12, a clearance slot 84 for receiving inner cutting member 110 of cutting member 18, and a cam surface 86 for interfacing with bushing 76. An o-ring 88 slidaby and fluidly seals nose member 16 to inner cutting member 110.

Alternatively, vitrectomy probe 10 may be formed with an actuating handle 14 have a similar geometry as shown in the Figures but with a continuous outer surface and without flexible appendages 14a. In this embodiment, cam member 72 is eliminated.

As described above, cutting member 18 preferably includes tubular outer cutter member 100 and tubular inner cutting member 110. Outer cutting member 100 has an inner bore 102, a closed end 104, a port 106 for receiving ophthalmic tissue, and cutting surfaces 108. Inner cutting member 110 has an inner bore 112, an open end 114, and a cutting surface 116. The ophthalmic tissue received by port 106 is preferably vitreous or membranes.

In operation, vitrectomy probe 10 is operatively coupled to a microsurgical system 198. More specifically, pneumatic port 22 is fluidly coupled to a pneumatic pressure source 200 via a fluid line 202, pneumatic port 24 is fluidly coupled to a pneumatic pressure source 204 via fluid line 206, and aspiration port 34 is fluidly coupled to vacuum source 208 via fluid line 209. Inner bore 112 and fluid line 209 are primed with a surgical fluid. Microsurgical system 198 also has a microprocessor or computer 210, which is electrically coupled to pneumatic pressure sources 200 and 204 via interfaces 212 and 214, respectively.

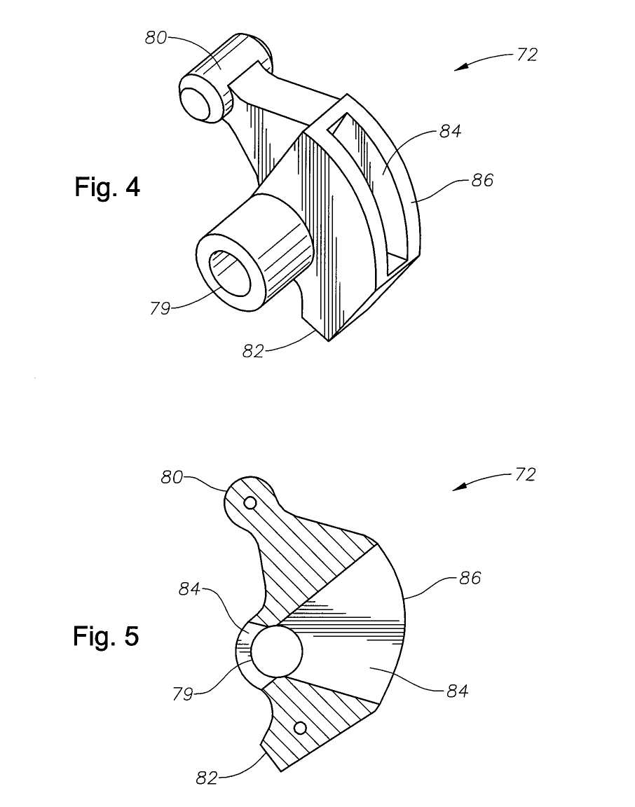

A surgeon inserts distal tip 20 into the posterior segment of the eye using a pars plana insertion. The surgeon selects a desired vacuum level for vacuum source 208. Ophthalmic tissue is aspirated into inner bore 112 via port 106. The surgeon selects a desired cut rate for probe 10 using microprocessor 210 and optionally a proportional control device (not shown), such as a foot controller. More specifically, microprocessor 210 uses pressurized gas sources 200 and 204 to create a cyclic pressure differential across diaphragm 28 so as to move center support 30, and thus inner cutting member 110, in a reciprocating manner at the desired cut rate. When the pressure provided to pneumatic port 22 is greater than the pressure provided to pneumatic port 24, inner cutting member 110 is moved toward distal tip 20 until open end 114 is past cutting surface 108, as shown in FIG. 6. This actuation closes port 106, allowing cutting surfaces 108 and 116 to cut the ophthalmic tissue within inner bore 112. The cut ophthalmic tissue is aspirated through inner bore 112, aspiration port 34, fluid line 209, and into a collection chamber (not shown). When the pressure provided to pneumatic port 24 is greater than the pressure provided to pneumatic port 22, inner cutting member 110 is moved away from distal tip 20, opening port 106 and allowing the further aspiration of ophthalmic tissue.

During actuation of inner cutting member 110, limiting surface 31 a of center support 30 contacts wall portion 33a of diaphragm chamber 26 to precisely end the cutting stroke. Limiting surface 3 lb of center support 30 contacts wall portion 33b of diaphragm chamber 26 to precisely end the return stroke. When limiting surface 31 a contacts wall portion 33a, cutting surface 116 of open end 114 of inner cutting member 110 is preferably disposed at or just past distal cutting surface 108 of outer cutting member 100. When limiting surface 31b contacts wall portion 33b, open end 114 is preferably disposed at or near proximal cutting surface 108 of outer cutting member 100. Such precision control of the actuation of inner cutting member 110 greatly increases the cutting efficiency of probe 10.

During actuation of inner cutting member 110, microprocessor 210 and actuating mechanism 13 provide a fixed and minimized amount of time that port 106 is open in each cut cycle of probe 10, which, for a given vacuum level or a given flow rate, in turn provides a fixed and minimized characteristic length (or characteristic volume) of cut ophthalmic tissue for all cut rates of probe 10. The fixed and minimized amount of time port 106 is open in each cut cycle of probe 10 is preferably about 0.5 to about 10 milliseconds, more preferably about 0.5 to about 6 milliseconds, more preferably about 0.5 to about 4 milliseconds, and most preferably about 0.5 to about 1 milliseconds. More specifically, microprocessor 210 uses pneumatic pressure source 204 to quickly open port 106, allowing ophthalmic tissue to flow into port 106 and inner bore 112. Microprocessor 210 then uses pneumatic pressure source 200 to quickly close port 106, cutting the ophthalmic tissue within inner bore 112. Microprocessor 210 then uses pneumatic pressure sources 200 and/or 204 to hold open end 114 of inner cutting member 110 near distal cutting surface 108 of port 106 until it is time to actuate inner cutting member 110 to begin the next cut cycle of probe 10 for a given cut rate.

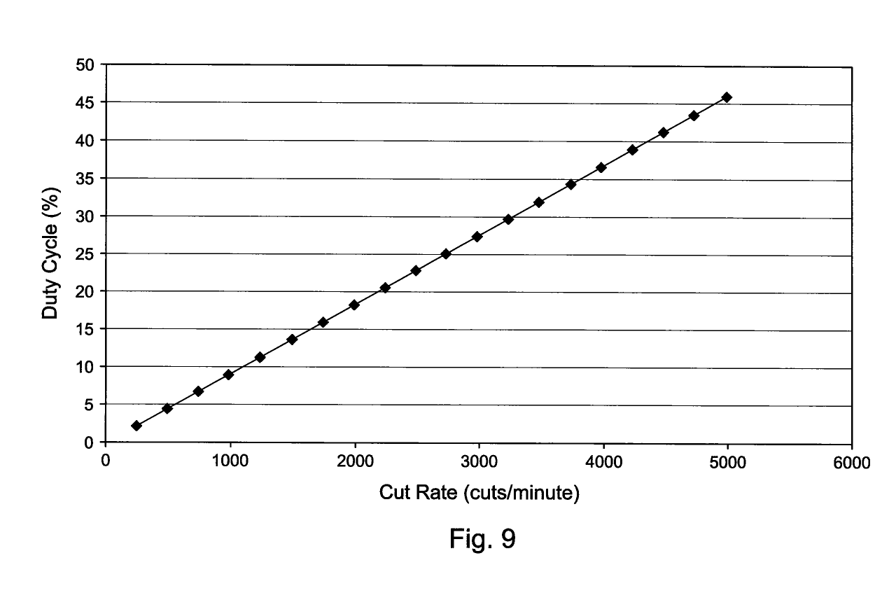

Referring to FIG. 8A, a characteristic length 300 of cut ophthalmic tissue 302 is shown during conventional operation of vitrectomy probe 10. The next piece of ophthalmic tissue to be cut by probe 10 is shown by reference numeral 304. In conventional operation of vitrectomy probe 10, characteristic length 300 is dependent on the cut rate of probe 10, the vacuum level applied to inner bore 112 of probe 10, and the physical properties of probe 10 such as the internal diameter of inner bore 112, in addition to the amount of time that port 106 is open during each cut cycle of probe 10. In conventional operation of probe 10, the amount of time that port 106 is open during each cut cycle of probe 10 is either fixed, but not minimized, for all cut rates of probe 10, or alternatively varies with the cut rate of probe 10. In contrast, FIG. 8B shows a characteristic length 306 of cut ophthalmic tissue that has been minimized according to the present invention. As the fibrils of both cut ophthalmic tissue 302 and ophthalmic tissue 304 are connected to other ophthalmic tissue and eventually to the retina, it has been discovered that characteristic lengths 300, 306 are an appropriate measure of retinal disruption during vitrectomy surgery. By minimizing characteristic length 306 for all cut rates of probe 10, disruption to the retina is minimized, and patient safety is maximized. This method of operation of vitrectomy probe 10 is particularly advantageous for management of delicate tissues near the retina and for membranes. FIG. 9 shows a preferred relationship between duty cycle and cut rate of probe 10 when probe 10 is operated according to the present invention. As used herein, “duty cycle” refers to the amount of time that port 106 is open during each cut cycle of probe 10 divided by the total amount of time in each cut cycle of probe 10.

From the above, it may be appreciated that the method of operation of a vitrectomy probe according to the present invention provides significant benefits over conventional methods of operation. It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A method of operating a vitrectomy probe, comprising the steps of:
providing a vitrectomy probe, said probe having:
a tubular outer cutting member with a port for receiving ophthalmic tissue and a tubular inner cutting member disposed within said outer cutting member; and
an actuating mechanism for reciprocating actuation of said inner cutting member so that said inner cutting member opens and closes said port and cuts said ophthalmic tissue disposed in said port;
providing a computer;
disposing said port in a posterior segment of an eye;
aspirating said ophthalmic tissue into said port; and
using said computer and said actuating mechanism to operate said probe over a range of cut rates so that an amount of time that said port is open during each cut cycle of said probe is fixed and minimized over all cut rates within said range of cut rates.

2. The method of claim 1 wherein said using step minimizes said amount of time to about 0.5 milliseconds to about 10 milliseconds.

3. The method of claim 1 wherein, for a given vacuum level or a given flow rate, said using step creates a characteristic length of said ophthalmic tissue cut in each cut cycle of said probe, said characteristic length being fixed and minimized over all cut rates within said range of cut rates.

4. The method of claim 1 wherein said ophthalmic tissue is vitreous.

5. The method of claim 1 wherein said ophthalmic tissue is a membrane.

6. The method of claim 1 further comprising the steps of:
providing a pneumatic pressure source;
fluidly coupling said pneumatic pressure source to said actuating mechanism;
operatively coupling said pneumatic pressure source to said computer; and
using said computer, said pneumatic pressure source, and said actuating mechanism to operate said probe over a range of cut rates so that an amount of time that said port is open during each cut cycle of said probe is fixed and minimized over all cut rates within said range of cut rates.

7. The method of claim 1 wherein said using step minimizes said amount of time to about 0.5 milliseconds to about 6 milliseconds.

8. The method of claim 1 wherein said using step minimizes said amount of time to about 0.5 milliseconds to about 4 milliseconds.

9. The method of claim 1 wherein said using step minimizes said amount of time to about 0.5 milliseconds to about 1 milliseconds.
